(19) United States
(12) Patent Application Publication
(10) Pub. No.: US 2011/0184313 A1
(43) Pub. Date: Jul. 28, 2011

(54) Cauterization Device and Method of Cauterizing

(75) Inventors: Yogesh B. Gianchandani, Ann Arbor, MI (US); Karthik Visvanathan, Ann Arbor, MI (US)

(73) Assignee: THE REGENTS OF THE UNIVERSITY OF MICHIGAN, Ann Arbor, MI (US)

(21) Appl. No.: 13/010,461

(22) Filed: Jan. 20, 2011

Related U.S. Application Data
(60) Provisional application No. 61/297,547, filed on Jan. 22, 2010.

(51) Int. Cl. A61B 10/02; A61B 18/04

(52) U.S. Cl. 600/567; 606/28

(57) ABSTRACT

A medical device such as a biopsy needle or probe has an integrated piezoelectric transducer. A power source is electrically coupled to the piezoelectric transducer. The power source is configured to generate a signal that causes the piezoelectric transducer to generate heat for cauterizing tissue. A signal analyzer receives a signal from the piezoelectric transducer, or from a sensor integrated into the biopsy needle or probe, to determine the extent of the cauterization.

### CROSS-REFERENCE TO RELATED APPLICATION

The present application claims the benefit of U.S. Provisional Application No. 61/297,547, entitled “CAUTERIZATION DEVICE AND METHOD OF CAUTERIZING,” filed on Jan. 22, 2010, which is hereby incorporated by reference herein in its entirety.

### STATEMENT REGARDING FEDERALLY SPONSORED RESEARCH OR DEVELOPMENT

This invention was made with government support under N66001-07-1-2006 awarded by Navy/SPAWAR, and under EECS 0734962 awarded by the National Science Foundation. The government has certain rights in the invention.

### BACKGROUND OF THE DISCLOSURE

1. Field of the Disclosure

The disclosure relates generally to biopsy needles and, more particularly, to biopsy needles capable of cauterizing the needle tract.

2. Brief Description of Related Technology

Needle aspiration biopsy is a diagnostic procedure used to investigate thyroid, breast, liver and lung cancers. Even though percutaneous biopsies are generally safe, there have been reports of potential risks such as deposition of viable tumor cells or “seeding” along the needle tract. The rate of seeding can vary from 5.1%-12.5%. Studies also suggest that post biopsy hemorrhage (bleeding) can be as high as 18.3%-23%. Further, this percentage can be higher for patients with cirrhosis and uncorrected coagulopathy. Infection is also a potential risk.

Past work had been limited to using radio frequency (RF) ablation of needle tracts. For instance, in one method, the outside of a biopsy needle, except for the last two centimeters, was coated with a thin layer of electrical insulation. A source of RF electrical power was then connected to the biopsy needle as it was withdrawn from the body, to provide electro-cauterization of the needle tract. Comparison of hemorrhage after liver and kidney biopsy, with and without ablation of the needle tract, was reported in W. F. Pritchard et al., “Radiofrequency cauterization with biopsy introducer needle,” J Vasc Intery Radiol, 15, pp. 183-187, 2004. Here, RF ablation by an introducer needle was employed as the ablation procedure. This study suggested that RF ablation reduces bleeding as compared to absence of RF ablation, in liver and kidney procedures, with mean blood loss reduced by 63% and 97%, respectively.

### SUMMARY OF THE DISCLOSURE

In an embodiment, a medical device such as a biopsy needle or probe has an integrated piezoelectric transducer. A power source is electrically coupled to the piezoelectric transducer. The power source is configured to generate a signal that causes the piezoelectric transducer to generate heat for cauterizing tissue.

In another embodiment, a medical procedure comprises inserting a medical device, such as a biopsy needle or a probe, into tissue of a patient. A piezoelectric transducer is integrated with the medical device. A power source electrically coupled to the piezoelectric transducer is used to cause the piezoelectric transducer to generate heat to cauterize tissue. Then, the medical probe is extracted.

In still another embodiment, a medical device such as a biopsy needle or probe has an integrated piezoelectric transducer. The piezoelectric transducer is connected to a power source that causes the piezoelectric transducer to generate heat to cauterize tissue. A control unit coupled to the power source monitors a signal from the medical device and controls the power source accordingly.

In another embodiment, a medical device such as a biopsy needle or probe has an integrated piezoelectric transducer. The piezoelectric transducer is electrically coupled to a power source and a servo. The servo is mechanically coupled to the medical device. A control unit, electrically coupled to both the servo and the power source, operates to control one or both of the servo and the power source according to a signal received from the piezoelectric transducer and/or the sensor.

### BRIEF DESCRIPTION OF THE FIGURES

FIG. 1 is a diagram of a biopsy system in accordance with an embodiment;

FIGS. 2A-2C are diagrams of several embodiments in which one or more piezoelectric transducers are integrated with a biopsy needle;

FIG. 3 is a diagram of an embodiment in which a plurality of piezoelectric transducers are integrated with a biopsy needle;

FIG. 4 is a graph of simulation results for variation of temperature as a function of distance from a needle corresponding to the embodiments illustrated in FIGS. 2A-2C;

FIG. 5A is a diagram of a model circuit for predicting variation in impedance characteristics of PZT;

FIG. 5B is a graph of analytical modeling results for variation of anti-resonance frequency of a modeled biopsy needle tip when the tip is in air, in tissue before cauterization, and in tissue after cauterization;

FIG. 6A is a diagram illustrating an example process for fabricating lead zirconate titanate (PZT) discs for use as a transducer;

FIG. 6B is a diagram illustrating an example process for mounting a piezoelectric sensor to a medical device such as a biopsy needle or probe;

FIG. 7 is a photograph of an embodiment of a biopsy needle having an array of PZT discs integrated thereto;

FIG. 8 is a graph of thermal efficiency and impedance of a PZT disc as a function of frequency at mode 2, where the PZT disc is bonded to a brass substrate using epoxy;

FIG. 9 is a graph of temperature attained by a PZT disc and conductance as a function of frequency of excitation at mode 2, where the PZT disc is bonded to a brass substrate using epoxy;

FIG. 10 is a graph of thermal efficiency and coupling factor for various mode shapes observed in a PZT disc bonded to a brass substrate using epoxy;

FIGS. 11A and 11B are graphs of the temperature measured at different distances and directions from a needle for the radial and thickness mode resonances, respectively;

FIGS. 12A and 12B are graphs showing variation in the temperature generated at the surface of the needle for various input voltages (FIG. 11A) and input power (FIG. 11B);

FIGS. 13A and 13B are photographs of porcine tissue cauterized using a biopsy needle such as illustrated in FIG. 7;

FIG. 14A is a graph showing measured variation of anti-resonance frequency and peak impedance magnitude for a needle in air, in tissue before cauterization, and in tissue after cauterization;

FIG. 14B is a graph showing measured variation of anti-resonance frequency with temperature in thr range used for cauterization;

FIG. 15 is a flow diagram of a method for obtaining a biopsy according to an embodiment; and

FIG. 16 is a block diagram of an embodiment of an apparatus for performing a servo-controlled biopsy and/or cauterization procedure.

### DETAILED DESCRIPTION OF PREFERRED EMBODIMENTS

Ultrasonic heating using piezoceramics holds significant promise as a tool for tissue cauterization. In some embodiments, ultrasonic heating using piezoceramics can be combined with ultrasonic tissue density measurements for determining completion of tissue cauterization. In an embodiment, heat generation in 3.2 mm diameter lead zirconate titanate (PZT) discs is used for biological tissue cauterization. In an embodiment, an array of 200 μm diameter bulk micromachined PZT transducers integrated with a 20-gauge biopsy needle provides for cauterization of the needle tract. In another embodiment, a single PZT transducer is utilized. In other embodiments, a single PZT transducer or an array of PZT transducers are mounted to a medical instrument other than a biopsy needle (such as a probe), or to a needle other than a 20-gauge biopsy needle, to provide for fine tissue cauterization.

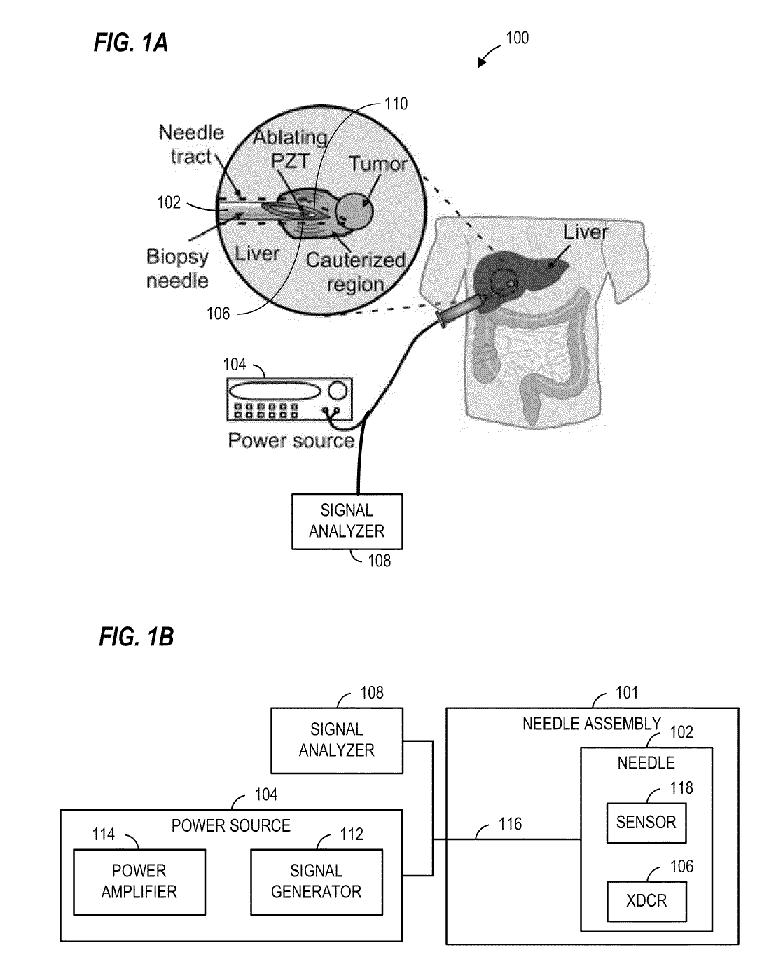

FIGS. 1A and 1B are, respectively, a diagram and a block diagram of an embodiment of a biopsy system 100. The system 100 includes a biopsy needle assembly 101 which includes a biopsy needle 102. One or more piezoelectric transducers 106 are integrated with the needle 102 proximate to a tip 110 of the biopsy needle 102. A link 116 electrically couples the one or more piezoelectric transducers 106 to a power source 104. The power source 104 may include a signal generator 112 and a power amplifier 114. In an embodiment, each of the one or more piezoelectric transducers 106 may be a PZT disc, each PZT disc having at least two resonant modes with corresponding resonant frequencies: a radial mode and a thickness mode. In this embodiment, the power source 104 generates an electrical signal that includes a sinusoidal component corresponding to the radial mode resonance and a sinusoidal component corresponding to the thickness mode resonance. If the system uses multiple PZT discs, and if different PZT discs have different resonance frequencies, the signal generated by the power source 104 may have signal components corresponding to the different resonance frequencies. The signal generated by the power source 104 may concurrently include multiple different resonance frequency components, or the power source 104 may alternately generate different resonance frequency components at different times (i.e., time multiplexing).

In an embodiment, the biopsy needle assembly 101 and, in particular, the biopsy needle 102 may include one or more sensors 118 integrated with the biopsy needle 102 and proximate to the tip 110. The one or more sensors 118 may be utilized to determine the extent of cauterization. For example, the one or more sensors 118 may comprise one or piezoelectric sensors. In this embodiment, the system 100 may include a signal analyzer 108, for example, an impedance analyzer, electrically coupled to the one or more sensors 118. The signal analyzer 108 may determine one or more resonance frequencies of the one or more piezoelectric sensors 118. As described in U.S. patent application Ser. No. 11/625,801, entitled “In-situ Tissue Analysis Device and Method,” filed on Jan. 22, 2007, which is hereby incorporated by reference herein, the resonance frequency of a piezoelectric sensor changes depending on the density of the tissue proximate to the piezoelectric sensor. Additionally, cauterized tissue has a different storage modulus than uncauterized tissue. Thus, the one or more piezoelectric sensors 118 can be utilized to determine the extent of cauterization (e.g., the depth and/or degree of cauterization of tissue in contact with the sensor/needle surface). In particular, the signal analyzer 108 may be utilized to monitor the resonance frequency of a piezoelectric sensor 118 to determine the extent of cauterization.

In another embodiment, the one or more piezoelectric transducers 106 used for cauterization are also configured for use as a sensor 118 to sense the degree of cauterization. For example, the piezoelectric transducers 106 can be utilized as sensors as described in U.S. patent application Ser. No. 11/625,801. As also described in U.S. patent application Ser. No. 11/625,801, in an embodiment, the piezoelectric transducers 106 and/or the sensors 118 aid in guiding the needle tip 110 to a target tissue (e.g., a tumor) by, for example, sensing changes in a property of a tissue which changes indicate a tissue boundary (e.g., the boundary between a tumor and the tissue in which the tumor is located).

In an embodiment, the biopsy needle 102 is a fine needle aspiration biopsy needle. For example, the needle 102 may be a 20-gauge needle, a 22-gauge needle, or a 25-gauge needle. In another embodiment, the needle is a non-needle probe. The non-needle probe may be used, for example, to cauterize or ablate target tissue (e.g., a tumor), which may be detected by the sensor 118. In still another embodiment, the needle is not a biopsy needle, but could be, for example, an injection needle. In the latter case, the injection needle may be used to deliver an injected substance to the target tissue, which may be detected using the sensors 118.

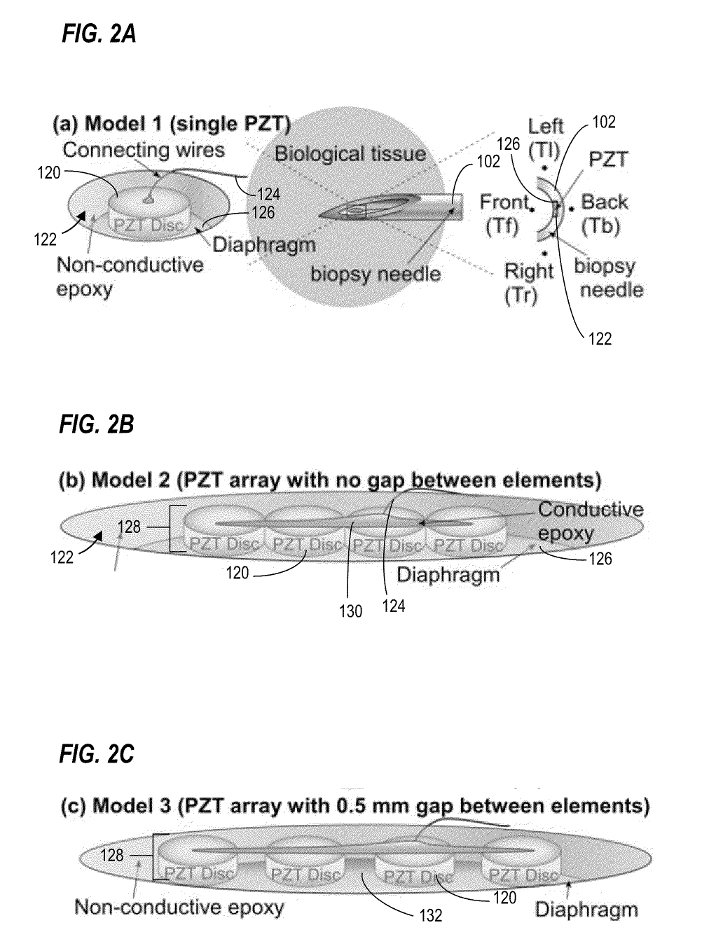

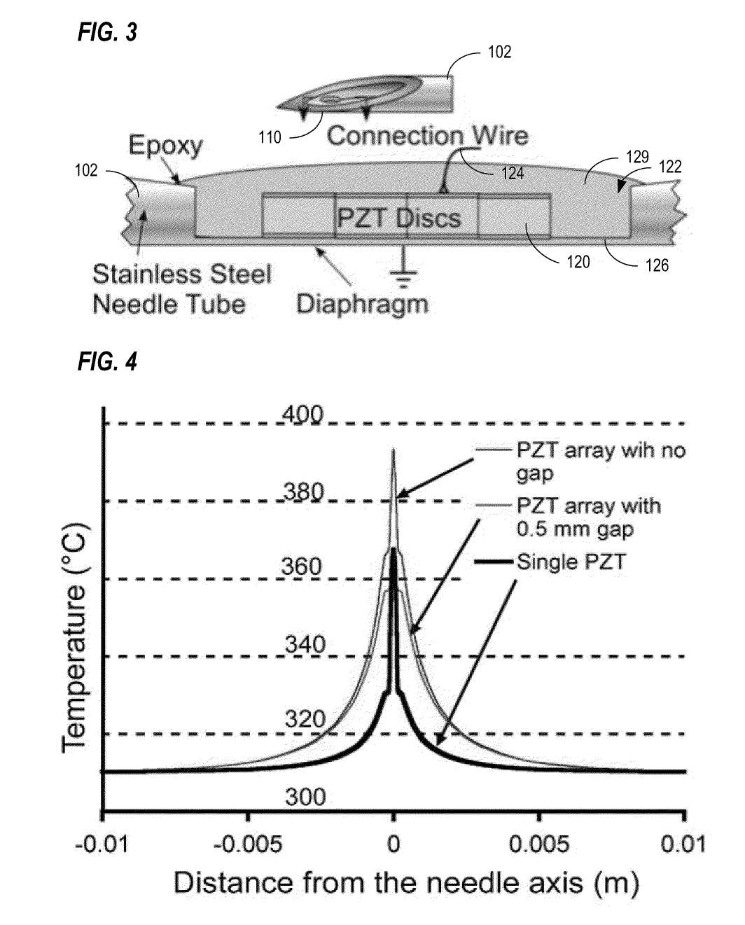

FIG. 2A illustrates one embodiment of a piezoelectric transducer 106 integrated with a biopsy needle 102. The piezoelectric transducer 106 comprises a single PZT disc 120 mounted in a cavity 122 of the biopsy needle 102 located proximate to the tip 110 of the biopsy needle 102. A non-conductive epoxy 129 (see FIG. 3) is used to mount the PZT disc 120 within the cavity 122. One or more wires 124 are coupled to the PZT disc 120 and electrically couple the PZT disc 120 to the power source 104. A wall 126 of the cavity 122 forms a diaphragm of the transducer 106.

FIG. 2B illustrates another embodiment of a piezoelectric transducer 106 integrated with a biopsy needle 102. The piezoelectric transducer 106 comprises an array 128 of PZT discs 120 mounted in a cavity 122 of the biopsy needle 102 located proximate to the tip 110 of the biopsy needle 102. A non-conductive epoxy 129 (see FIG. 3) is used to mount the array 128 of PZT discs 120 within the cavity 122. A conductive epoxy 130 electrically couples the array 128 of PZT discs 120 together. One or more wires 124 are coupled to the array 128 of PZT discs 120 and electrically couples the array 128 of PZT discs 120 to the power source 104. A wall 126 of the cavity 122 forms a diaphragm of the transducer 106. In this embodiment, each of the PZT discs 120 in the array 128 is in contact with a neighbor PZT disc 120.

FIG. 2C illustrates another embodiment of a piezoelectric transducer 106 integrated with a biopsy needle 102. The embodiment of FIG. 2C is similar to the embodiment of FIG. 2B, except that a gap 132 exists between neighboring PZT discs 120 in the array 128.

FIG. 3 is another illustration of the embodiment of FIG. 2B.

Temperature Profile Model

An example 3D finite element model was developed to estimate the temperature profile in the tissues. Pennes' bioheat transfer model was used to model heat transfer in tissues. This model takes into account the cooling due to blood flow in tissues. The model is given by:

$${ρ}_{t} {c}_{t} \frac{\partial T}{\partial t} = ∇ k ∇ T + {ρ}_{b} {c}_{b} {ω}_{b} ( {T}_{b} - T ) + q \qquad ( 1 )$$

where ρₜ is the density of the medium, cₜ is the specific heat capacity, k is the thermal conductivity, T is the temperature, ρ_{b} is the density of blood, c_{b} is the specific heat capacity of blood, ω_{b} is the perfusion rate of the blood, T_{b} is the arterial blood temperature and q is the heat generation rate per unit volume due to ultrasound applicator.

In a biopsy needle embodiment, PZT heaters are significantly smaller than the size of the needle. Hence, in a biopsy needle embodiment, the heaters can be modeled as small spherical sources. The heat generation rate from the PZT heater is given by:

$$q = \frac{2 α {I}_{s} {r}_{0}^{2}}{{r}^{2}} {e}^{- 2 μ ( r - {r}_{0} )} \qquad ( 2 )$$

where α is the ultrasound absorption coefficient (Np·m⁻¹), I_{S} is the ultrasound intensity along the surface of the transducer (Wm⁻²), r is the radial distance from the center of the transducer and r₀ is the radius of the transducer. The term μ is the ultrasound attenuation and is taken equal to a under the assumption that all the attenuated acoustic energy is absorbed by the local medium. However, due to inefficiencies in the transducer, not all the electrical energy applied to it gets converted into acoustic energy. This unconverted energy is dissipated as heat within the transducer. For a given transducer efficiency, ν, the heat generation rate per unit volume within the transducer is given by:

$${q}_{app} = ( \frac{1 - v}{v} ) \frac{3 {I}_{s}}{{r}_{0}} \qquad ( 3 )$$

FIG. 4 is a graph of simulation results for variation of temperature as a function of distance from the needle for the three designs illustrated in FIGS. 2A-2C for an ultrasound intensity, I_{S}=90 Wcm⁻². The simulations were performed using a bioheat equation model in COMSOL Multiphysics 3.4. Three designs were considered in the simulations: single PZT disc 120, PZT array 128 (4 discs 120) with no gap 132 between elements, and PZT array 128 with 0.5 mm gap 132 between elements. All models comprised four major regions: PZT heater, epoxy surrounding the PZT heater, biopsy needle and biological tissue. The biological tissue was modeled using a 5 cm diameter sphere surrounding the needle 102. The needle 102 was modeled using a partial cylinder with inner and outer radii of 300 μm and 450 μm, respectively. The length of the needle 102 was 6 cm. For a single PZT design, a hole of 135 μm depth and 300 μm diameter was created to model the cavity 122 for placing the PZT heater. In the case of array design, a slot of 2000×300×135 μm³ was created in the needle 102. The material properties used in the simulations are shown in Table 1.

The cooling due to blood flow was considered only in the biological tissue region. The heat generation rate given in equation 2 was used in epoxy, needle and tissue regions. The heat generation rate given by equation 3 was used in the PZT region. The outer surface of the tissue and the far end tip of the needle (outside the tissue region) were maintained at 310 K and 300 K, respectively. In the simulations, transducer efficiency was assumed to be 0.52. FIG. 4 compares the simulation results for temperature variation as a function of distance from the needle. Simulations suggest that for an ultrasonic surface intensity (that is proportional to drive voltage) of 90 Wcm⁻², maximum temperature is attained by PZT array 128 with no gap 132 between the elements.

TABLE 1
Material properties used in the simulations

| | | |
|---|---|---|
| Density of tissue | 1050 | kgm⁻³ |
| Thermal conductivity of tissue | 0.51 | Wm⁻¹K⁻¹ |
| Specific heat capacity of tissue | 3639 | Jkg⁻¹K⁻¹ |
| Density of blood | 1000 | kgm⁻³ |
| Specific heat capacity of blood | 4180 | Jkg⁻¹K⁻¹ |
| Perfusion rate of blood | 15 × 10⁻³ | s⁻¹ |
| Arterial blood temperature | 310 | K |
| Thermal conductivity of needle | 44.5 | Wm⁻¹K⁻¹ |
| Density of needle | 7850 | kgm⁻³ |
| Specific heat capacity of needle | 475 | Jkg⁻¹K⁻¹ |
| Thermal conductivity of epoxy | 1.7 | Wm⁻¹K⁻¹ |
| Density of epoxy | 1060 | kgm⁻³ |
| Specific heat capacity of epoxy | 1000 | Jkg⁻¹K⁻¹ |
| Thermal conductivity of PZT | 1 | Wm⁻¹K⁻¹ |
| Density of PZT | 7700 | kgm⁻³ |
| Specific heat capacity of PZT | 350 | Jkg⁻¹K⁻¹ |

Electrical Impedance Model

In a needle 102 having one or more piezoelectric transducers 106 for cauterizing and/or ablating tissue, one or more sensors 118 in the needle 102 may detect changes in the impedance characteristics of the sensor 106 (e.g., one or more PZT discs 120) due to the cauterization.

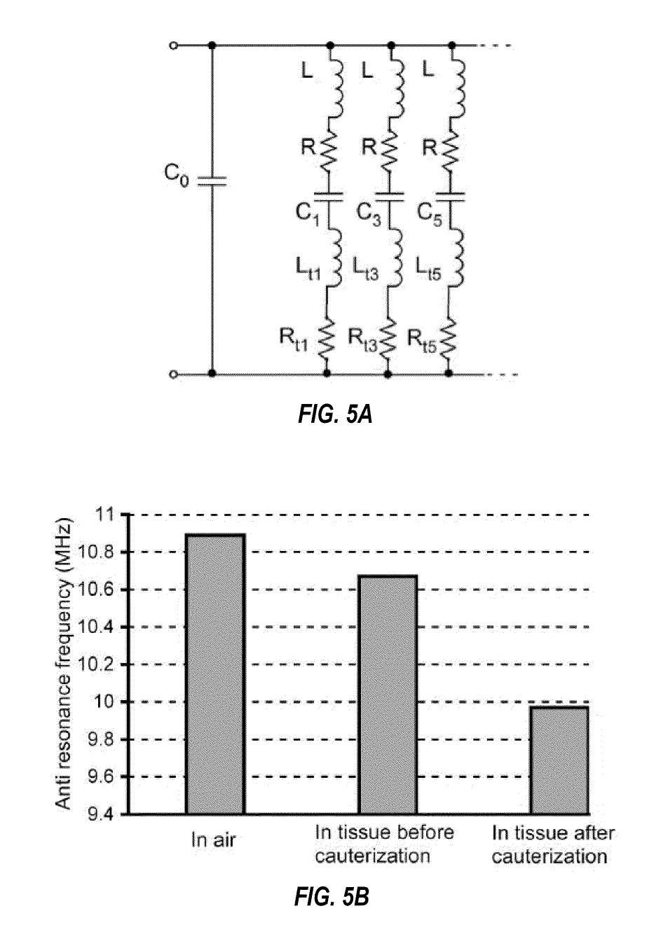

The resonance frequency and magnitude of the electromechanical impedance of a PZT-embedded structure depend on the density, elastic modulus and loss factor of the surrounding medium. The elastic modulus and loss factor in the tissue increases after ablation, thereby providing a method for monitoring tissue cauterization. A modified Butterworth-Van-Dyke (BVD) circuit model (see FIG. 5A) is used to predict the variation in impedance characteristics of the PZT disc 120 in air, and in tissue before and after cauterization. The circuit includes a static branch (C₀) and infinite motional branches (R, L, Cₙ) connected in parallel, with each motional branch corresponding to different resonance modes. The various resistors, capacitors and inductors in the circuit are:

$$\begin{matrix}{C}_{0} = \frac{ɛ A}{{t}_{0}} & ( 4 ) \\ L = \frac{1}{4 {π}^{2} {f}_{a 1}^{2} {C}_{1}} & ( 5 ) \\ {C}_{n} = \frac{8 {k}_{t}^{2} / {n}^{2} {π}^{2}}{1 - 8 {k}_{t}^{2} / {n}^{2} {π}^{2}} {C}_{0} & ( 6 ) \\ R = \frac{{η}_{0}}{{ρ}_{0} {v}_{0}^{2} {C}_{1}} ( \frac{f}{{f}_{a 1}} ) & ( 7 )\end{matrix}$$

where kₜ is the electro-mechanical coupling constant, η₀ is the viscosity of PZT layer, ρ₀ is the density of PZT, A is the area of PZT, v₀ is the acoustic velocity in PZT, t₀ is the PZT thickness and ∈ is the dielectric permittivity in PZT. The resonance frequency, fₘ (at minimum impedance), and the anti-resonance frequency, fₐₙ (at maximum impedance), are given by:

$$\begin{matrix}{f}_{an} = \frac{1}{2 π \sqrt{{LC}_{n}}} & ( 8 ) \\ {f}_{rn} = \frac{1}{2 π \sqrt{{LC}_{n} \frac{{C}_{0}}{{C}_{0} - {C}_{n}}}} & ( 9 )\end{matrix}$$

The effect of tissue loading is modeled by adding the resistor Rₜₙ and inductor Lₜₙ to the motional branches of the circuit. For a semi-infinite viscoelastic medium Rₜₙ and Lₜₙ are given by:

$$\begin{matrix}{R}_{tn} = {\frac{n π}{4 {k}_{t}^{2} ω {C}_{0} {Z}_{q}} [ \frac{{ρ}_{t} ( \vert G \vert + {G}^{′} )}{2} ]}^{0.5} & ( 10 ) \\ {L}_{tn} = {\frac{n π}{4 {k}_{t}^{2} {ω}^{2} {C}_{0} {Z}_{q}} [ \frac{{ρ}_{t} ( \vert G \vert - {G}^{′} )}{2} ]}^{0.5} & ( 11 )\end{matrix}$$

where G=G′+iηω, Z_{q}=\sqrt{E₀ρ₀}, E₀ is the Young's modulus of PZT, ρₜ is the tissue density, ω is the operation frequency, G′ is the tissue storage modulus, η is the loss factor in tissue, and Z_{q} is the PZT acoustic impedance. Table 2 lists the material properties used in the model. The fundamental anti-resonance frequency, which is the mode to be used for experiments, when the biopsy needle tip is in air, and in tissue before cauterization and after cauterization, is shown in FIG. 5B. Analytical modeling shows that the fundamental anti-resonance frequency decreases by 0.65 MHz after cauterization.

TABLE 2
Material properties used in the BVD analytical model

| Normal Tissue | | |
|---|---|---|
| Density, ρₜ | 1054 | kgm⁻³ |
| Storage modulus, G′ | 5500 | Pa |
| Loss factor, η | 13 | Pa · s |
| Cauterized tissue | | |
| Storage modulus, G′ | 3700 | Pa |
| Loss factor, η | 230 | Pa · s |
| PZT-5A | | |
| Young's modulus, E₀ | 5.2 × 10¹⁰ | Pa |
| Density, ρ₀ | 7800 | kgm⁻³ |

| | |
|---|---|
| Coupling constant, Kₜ | 0.72 |
| Relative dielectric constant | 1800 |

Experimental Device Design and Fabrication

In an experimental device, PZT discs were fabricated from PZT-5A material. This material has a Curie temperature of 350° C., which is greater than the target temperature of 70-100° C. (ΔT=33−63° C.). Circular shaped PZT devices were used because for a given volume device, these generate higher temperature rise per unit voltage as compared to square and rectangular devices.

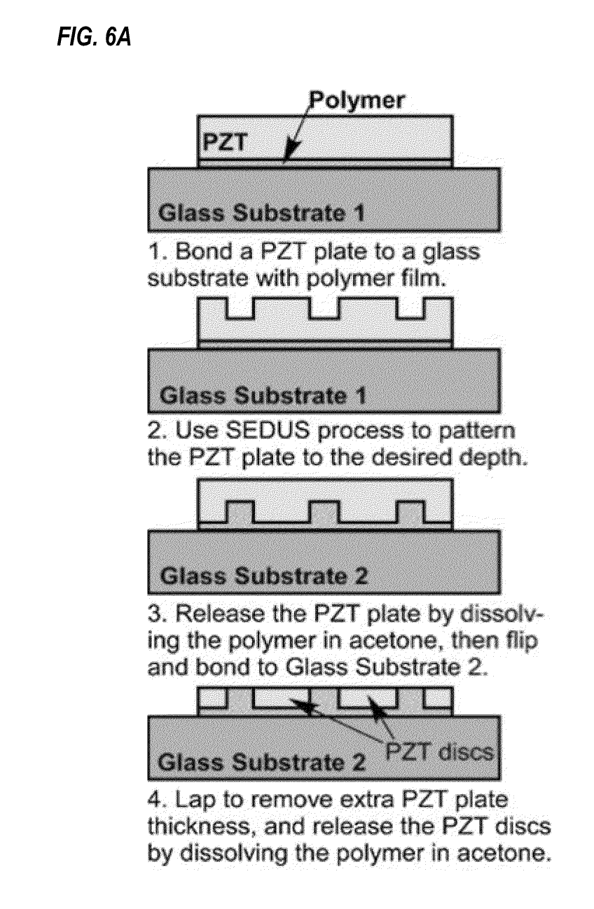

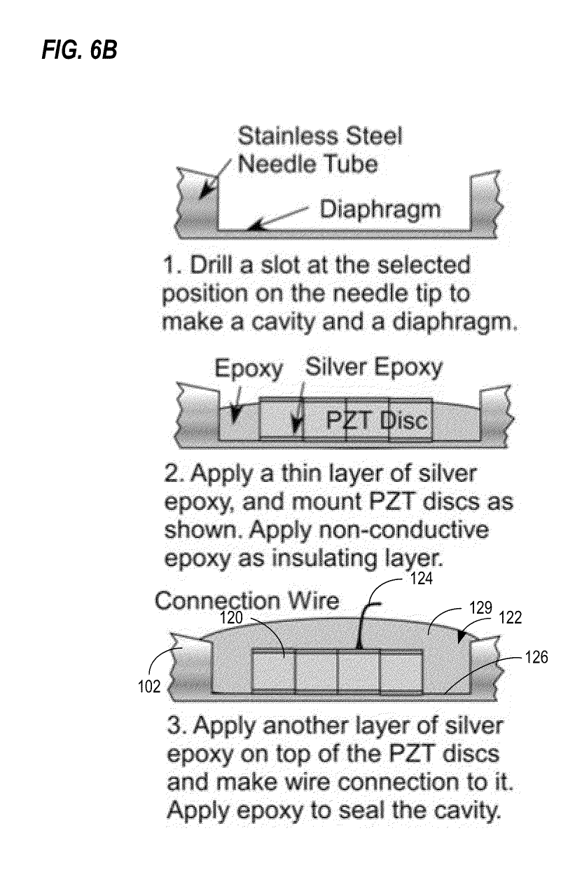

FIG. 6A is a diagram illustrating an example process for fabricating PZT discs. In this embodiment, the PZT discs (diameter=200 μm; thickness=70-100 μm) were fabricated using an ultrasonic micromachining process (USM). The USM tools were fabricated using micro electro-discharge machining (μ-EDM) of stainless steel. The pattern was then transferred to the PZT-5A plate using USM with tungsten carbide slurry. The patterned PZT discs were released by lapping from behind. Finally, a 500 nm thick gold layer was sputtered to form the electrodes. The sides of the discs were covered with a thin layer of photoresist to prevent shorting of the two electrodes during sputtering.

FIG. 6B is a diagram illustrating an example process for mounting a piezoelectric sensor 118 to a medical device such as a biopsy needle 102 or probe. The PZT discs 120 are integrated into a recess or cavity 122 (2000×300×135 μm³) cut into the needle 102 or probe (such as a 20 gauge needle) using μ-EDM or another suitable process. In a biopsy needle application, this prevents the discs 120 from blocking the path for acquiring tissues during the biopsy process. The thin diaphragm left behind in the wall 126 of the needle 102 after the formation of the cavity 122 also reduces the heat loss due to conduction through the needle 102. The PZT discs 120 were surrounded by non-conductive epoxy 129 in order to provide a highly damping medium for heat generation as well as reduce heat loss due to conduction. Flexible copper wire within lumen provided power to the top electrode while the needle provided the ground return path.

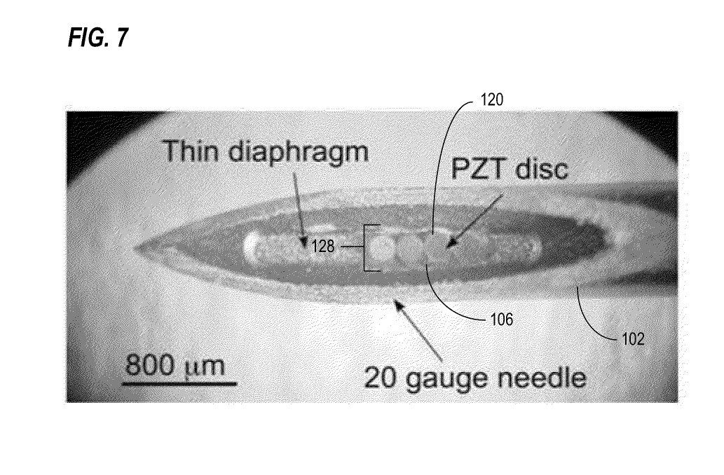

FIG. 7 is a photograph illustrating a piezoelectric transducer 106 (an array 128 of PZT discs 120) integrated with a biopsy needle 102.

Operating Frequency

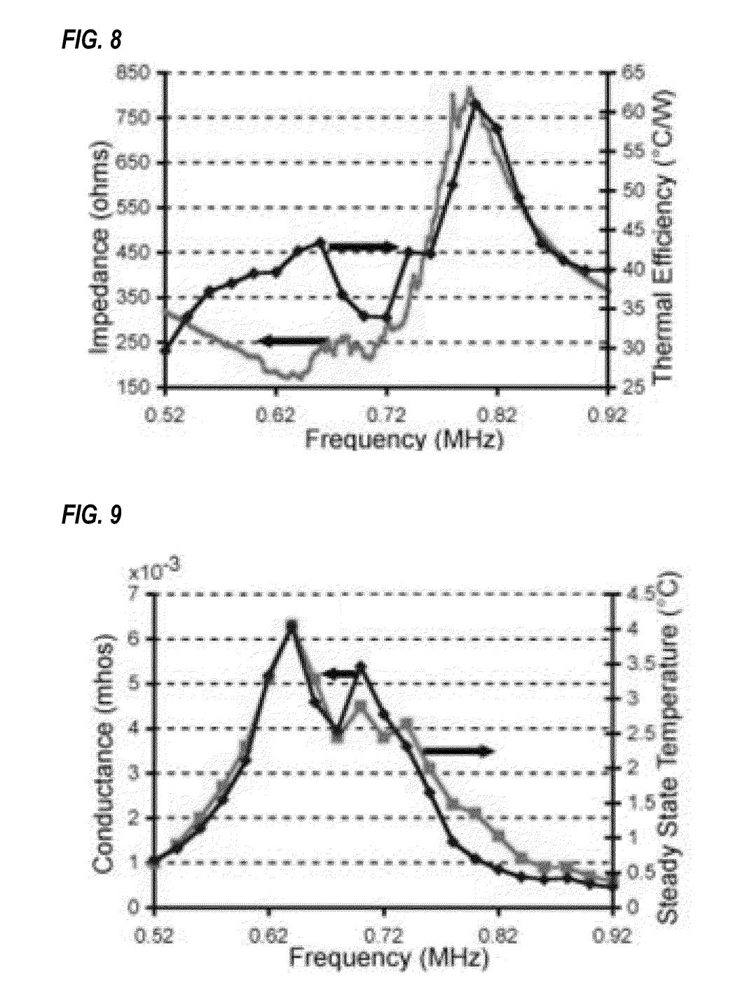

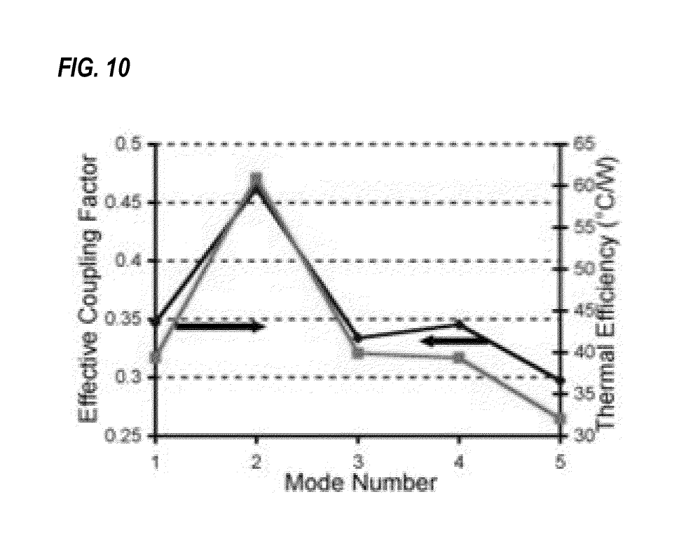

PZT discs may be characterized to determine the operating frequency that provides maximum thermal efficiency. FIG. 8 is a graph of thermal efficiency and impedance of a PZT disc 120 as a function of frequency at mode 2, where the PZT disc 120 is bonded to a brass substrate using epoxy. FIG. 8 suggests that the PZT disc 120 may attain a maximum efficiency at its anti-resonance (maximum impedance) frequency. This is believed to be due to a minimum of parasitic losses, as the current flowing through the system is a minimum for a given voltage. The variation of steady state temperature was also studied. FIG. 9 is a graph of temperature attained by a PZT disc 120 and conductance as a function of frequency of excitation at mode 2, where the PZT disc 120 is bonded to a brass substrate using epoxy. FIG. 9 suggests that the change in temperature may be a maximum at the frequency of a maximum conductance (minimum impedance). Hence, when selecting the frequency, there may be a trade-off between maximum temperature and maximum efficiency, depending on the application. The thermal efficiencies of various resonance modes were also studied with a PZT disc 120 bonded to a brass substrate using epoxy. It was observed that the thermal efficiency is proportional to the effective coupling factor (k_{eff}) of each mode (FIG. 10). The effective coupling factor is defined as:

$${k}_{eff} = {( \frac{{f}_{ar}^{2} - {f}_{r}^{2}}{{f}_{ar}^{2}} )}^{0.5}$$

(12)
where fₐᵣ is the anti-resonance frequency and fᵣ is the resonance frequency. For the case in which a PZT disc 120 is bonded to a brass substrate, mode 2 was observed to be suitable.

Experimental Results—Temperature Profile

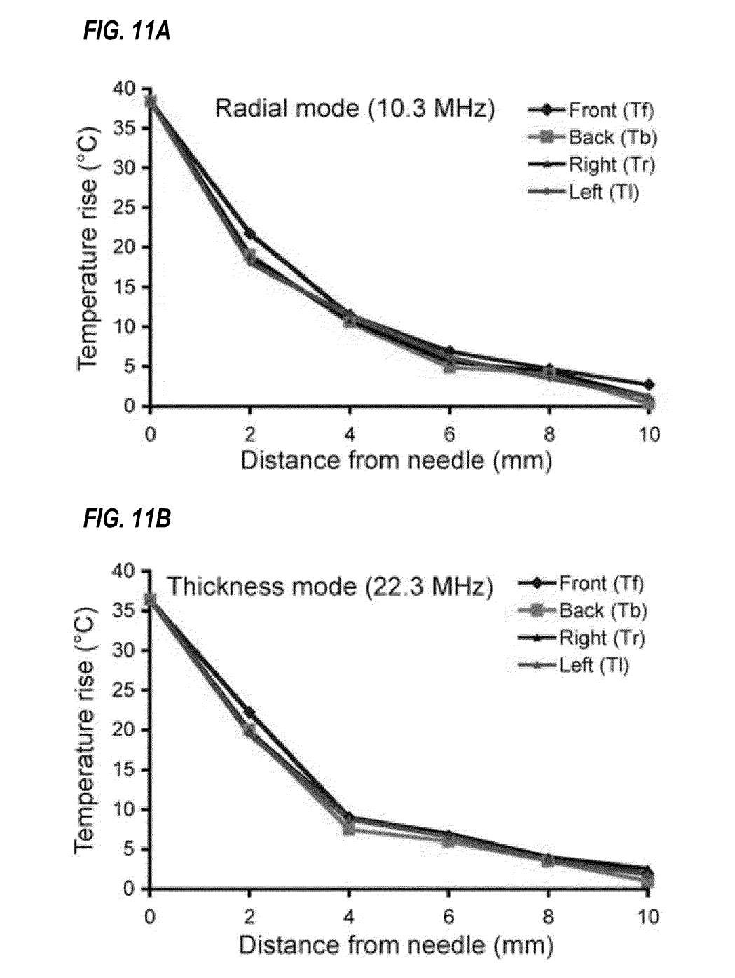

The temperature profile generated by a first experimental biopsy tool was measured at two resonance modes: the radial mode (10.3 MHz) and the thickness mode (22.3 MHz). PZT discs 120 were actuated using a sinusoidal wave at the respective resonance frequencies using the signal generator 112 amplified using the power amplifier 114. The temperature was measured using a K-type thermocouple (not shown) read using a digital thermometer. The experiments were performed by inserting the needle 102 of the needle assembly 101 into porcine tissue samples. FIGS. 11A and 11B are graphs of the temperature measured at different distances and directions from the needle 102 for the radial and thickness mode resonances, respectively. The temperature distribution is similar in all directions for both resonance modes. This indicates uniform cauterization in the surrounding region.

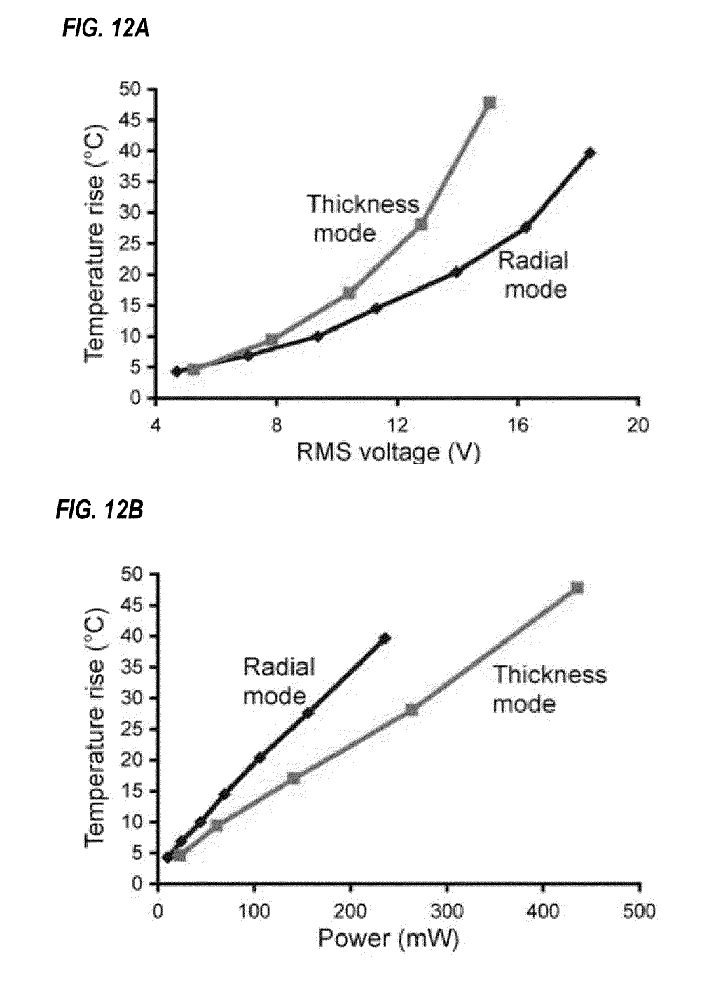

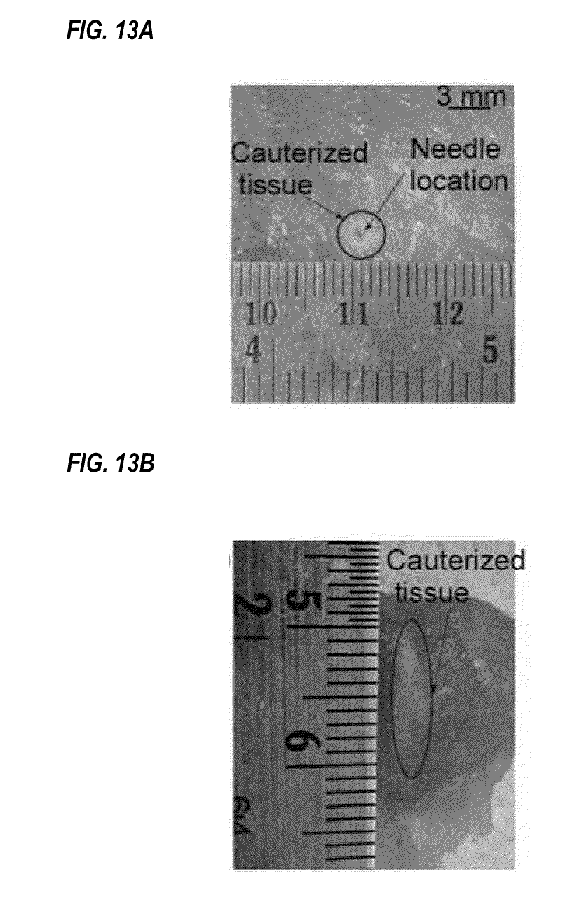

FIGS. 12A and 12B are graphs showing variation in the temperature generated at the surface of the needle 102 for various input voltages (FIG. 12A) and input powers (FIG. 12B). The temperature rise at the surface of needle 102, in both resonance modes, for varying input voltage is shown in FIG. 12A. The needle surface exceeded the minimum target temperature rise of 33° C. for an applied voltage of 17 VRMS and 14 VRMS for radial and thickness modes, respectively. FIG. 12B compares the temperature rise generated at the surface of the needle 102 for various input powers for the two modes. The plot suggests that the target temperature rise of 33° C. was achieved for input power of 236 mW and 325 mW, respectively. This difference is believed to be mainly due to the higher electromechanical impedance of the PZT discs 120 at lower operating frequency. FIGS. 13A (top view) and 13B (cross-section) are photographs of the cauterized porcine tissue for an applied voltage of 14 VRMS at 22.3 MHz. The radius of tissue cauterization is 1-1.25 mm beyond the perimeter of needle 102. This ensures minimal damage to the surrounding healthy tissue.

Experimental Results—Electrical Impedance

Additional experiments were conducted by inserting a second experimental biopsy tool into a porcine tissue sample. The porcine tissue sample was cauterized by actuating the PZT discs 120 with an RMS voltage of 14 V as their fundamental anti-resonance frequency of 9.6 MHz. The impedance characteristics of the PZT discs 120 were measured using an Agilent 4395A impedance analyzer. All impedance measurements were conducted at room temperature, unless otherwise stated.

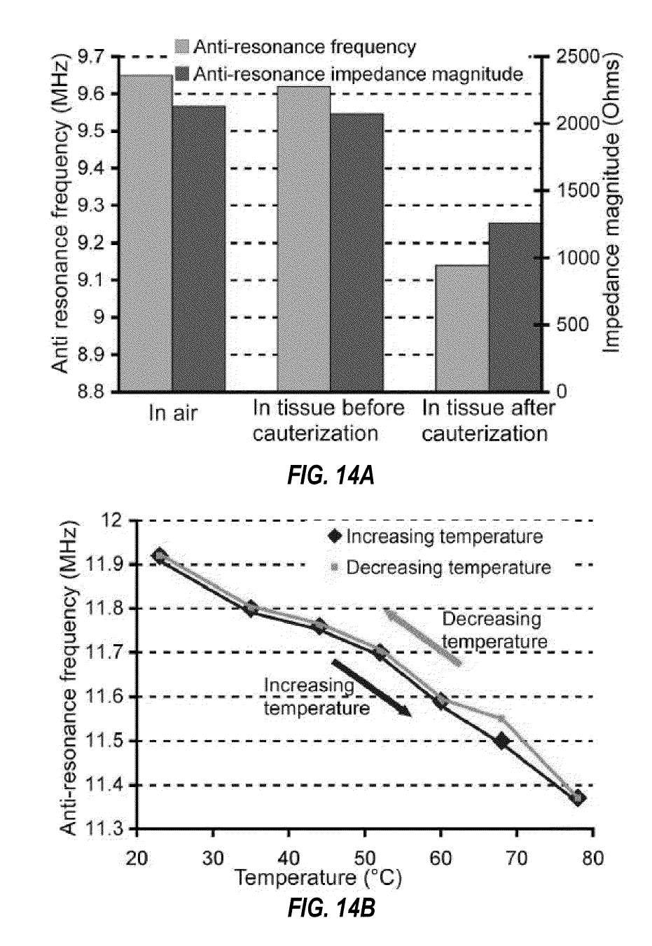

FIG. 14A shows the variation of the impedance characteristics of the PZT transducer 106 for the following three cases: biopsy needle tip 110 in air, and in tissue before and after cauterization. The fundamental anti-resonance frequency (fₐ₁) of the PZT discs 120 was used for monitoring of cauterization. When the biopsy needle was inserted into the tissue, fₐ₁ dropped from 9.66 MHz to 9.61 MHz. After cauterization, fₐ₁ and the peak impedance magnitude further decreased by 0.6 MHz and 900 ohms, respectively (FIG. 14A). This decrease matches to that predicted by the analytical model and can be used to monitor the progress of cauterization.

The variation in fₐ₁ was also measured with temperature varied in the range for cauterization while the needle tip stayed in air. Even though fₐ₁ decreased (from 11.92 MHz to 11.38 MHz) with increasing temperature (from 22° C. to 78° C.), it was observed that fₐ₁ returned to its initial value when the needle 102 was cooled down to room temperature (FIG. 14B). As the readings in FIG. 14A were all made at the same room temperature, additional correction is unnecessary.

Cauterizing Tissue

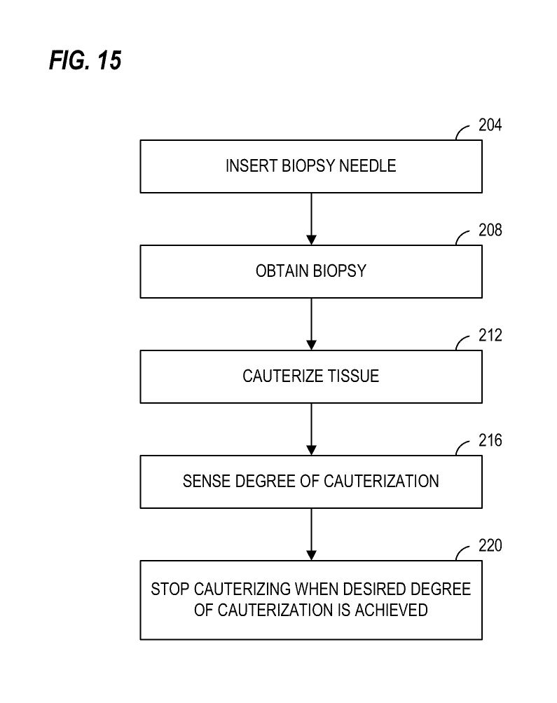

FIG. 15 is a flow diagram of an embodiment of a method for utilizing a system such as described with reference to FIG. 1. At block 204, the biopsy needle 102 is inserted into position to obtain a biopsy. If piezoelectric sensors 118 are integrated with the biopsy needle 102 as described in U.S. patent application Ser. No. 11/625,801, the piezoelectric sensors 118 may be used to guide the biopsy needle 102 to the correct position as described in U.S. patent application Ser. No. 11/625,801. At block 208, the biopsy needle 102 is used to obtain a biopsy.

At block 212, tissue is cauterized using the one or more piezoelectric transducers 106. In an embodiment, block 212 may comprise providing signals to the one or more piezoelectric transducers 106 having signal components corresponding to resonant frequencies of the one or more piezoelectric transducers 106. Block 212 may be performed while the needle 102 is stationary and/or while the needle 102 is slowly being withdrawn so that the needle tract is cauterized along the length of the tract.

If the system includes a sensor 118, at block 216, the sensor 118 is utilized to determine an extent of cauterization. In an embodiment, a piezoelectric sensor 118 is utilized to sense differences in the density of tissue proximate to the sensor, which differences indicating a degree of cauterization. At block 220, cauterization is stopped when a desired degree of cauterization is achieved. In an embodiment, the piezoelectric transducer or transducers 106 receiving the signals and cauterizing the tissues may be utilized to determine an extent of cauterization. The transducers 106 and/or the sensors 118 may be used to determine an extent of cauterization by analyzing, for example with an impedance analyzer, the anti-resonance frequency and/or impedance magnitude of the transducers 106 and/or the sensors 118.

The blocks 212 and 216 may be performed alternately. For example, a time duration of cauterization may occur followed by a time duration of sensing, and the alternation repeating until the desired degree of cauterization is achieved. The blocks 212 and 216 may be performed while the needle is stationary and/or while the needle is slowly being withdrawn.

Automation

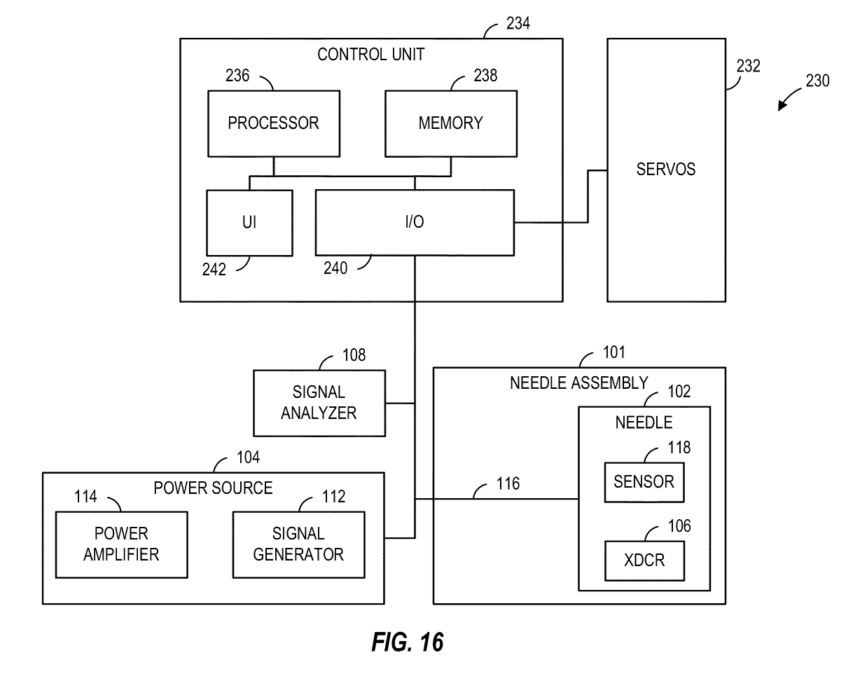

In some embodiments, the system 100 described above may be integrated into an automated system for performing a biopsy and/or for performing a cauterization process. FIG. 16 depicts a block diagram of a system 230 for performing a servo-controlled biopsy and/or cauterization procedure. The system 230 generally includes the components 101-118 of the system 100, as described with reference to FIG. 1B. Additionally, the system 230 includes one or more servos 232 and a control unit 234.

The servos 232 are mechanically coupled to the needle assembly 101 to manipulate the needle assembly 101. In some embodiments, the system 230 includes five servos 232 that allow the system 230 to manipulate the needle 102 with five degrees of freedom. For example, such a system 230 may move the needle along x- and y-axes orthogonal to the length of the needle 102 and to each other, along a z-axis aligned with the length of the needle 102 and orthogonal with each of the x- and y-axes (e.g., into and out of the patient), and may pivot the needle 102 about the x- and y-axes. Of course, in other embodiments, this degree of flexibility may be unnecessary and fewer servos 232 may be used. Minimally, a single servo 232 may be employed to move the needle 102 in a direction aligned with the length of the needle 102.

The system 230 may employ a control unit 234 to provide control signals to the servos 232. The control unit 234 includes a processor 236, a memory device 238, an input/output (I/O) interface 240, and a user interface 242. The control unit 234 may be electrically coupled to one or both of the signal analyzer 108 and the power source 104 via the I/O interface 240. The control unit 234 may also be electrically coupled to the servos 232 via the I/O interface 240. The processor 236 may execute one or more sets of instructions (e.g., programs, algorithms, etc.) stored in the memory device 238. The sets of instructions, or routines, stored in the memory device 238 may include a routine for allowing a user (e.g., a doctor, technician, etc.) to adjust a position of the needle 102 (e.g., by causing movement of the servos 232) through the user interface 242 prior to executing an automated procedure. One routine may allow the user to set parameters for the automated procedure. A routine may also operate to cause the control unit 234 to transmit a signal to the power source 104. The transmitted signal may perform a control action on the signal generator 112 or the power amplifier 114. For example, the transmitted signal may configure either or both of the signal generator 112 and the power amplifier 114 according to parameters entered through the user interface 242 by the user. Parameters may include the waveform parameters (e.g., voltage, waveform shape, frequency, etc.) and amplification factors for the signal transmitted to the transducer 106. Further, in some embodiments, a routine may cause the control unit 234 to send and/or receive one or more signals from the signal analyzer 108. The routine may cause the control unit 234 to configure the signal analyzer 108 to receive a signal from the sensor 118 or the transducer 106 and to determine whether the tissue in contact with the needle 102 has been adequately cauterized. At the same time, a routine may cause the control unit 234 to operate the servos 232 and/or adjust (e.g., reconfigure) one or more parameters of the power source 104 according to the determination of the signal analyzer 108. In one embodiment, a routine causes the control unit 234 to configure the signal analyzer 108 to receive and analyze a signal from a sensor 118 to determine when the tip 110 of the needle 102 has crossed a tissue boundary, for example, to prevent cauterization of certain tissue, or to guide the needle 102 to a target tissue. Of course, functionality of the one or more of the routines described above may be combined into fewer routines and/or separated into more routines.

While the control unit 234 is depicted in FIG. 16 as separate from the signal analyzer 108 and the power source 104, in some embodiments, one or more of the control unit 234, the signal analyzer 108, and the power source 104 may be contained within a single physical housing. In an embodiment, the control unit 234 is a personal computer or workstation (not shown) configured with one or more special-purpose devices designed to installed on the personal computer or workstation. The special purpose devices can include signal generator card, a power amplifier card, a digital I/O card, a signal analyzer card, etc., such as those sold by National Instruments, of Austin, Tex.

Although devices and techniques described above were in the context of biopsies, one of ordinary skill in the art will recognize that these cauterization devices and techniques can be utilized in other contexts as well. For example, a probe device could be used to cauterize a tumor or growth, or to stop source of bleeding. Similarly, one or more transducers, and optionally one or more sensors, could be mounted proximate to some other surgical tool to permit cauterization and optionally measuring the degree of cauterization using the surgical tool.

Properties or changes in properties sensed by the sensor(s) could be indicated to a physician, technician, etc., in a variety of ways. For example, properties or changes in properties could be indicated visually, audibly, with force feedback, etc. A computing device could be communicatively coupled to the sensors and/or to an interface device or devices (which is in turn communicatively coupled to the sensor(s)). The communication device could generate indications based on the properties or changes in properties sensed by the sensor(s).

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding only, and no unnecessary limitations should be understood therefrom, as modifications within the scope of the invention may be apparent to those having ordinary skill in the art.

We claim:

1. An apparatus, comprising:
a elongated device for insertion into living tissue, the elongated device comprising one of a probe or a needle;
a piezoelectric transducer integrated with the elongated device; and
a power source electrically coupled to the piezoelectric transducer, the power source configured to generate a signal that causes the piezoelectric transducer to generate heat for cauterizing tissue.

2. An apparatus according to claim 1, wherein the elongated device is a biopsy needle.

3. An apparatus according to claim 1, further comprising a signal analyzer electrically coupled to the piezoelectric transducer, the signal analyzer configured to provide a measurement indicative of an extent of cauterization sensed by the piezoelectric transducer.

4. An apparatus according to claim 3, further comprising:
one or more servos mechanically coupled to the elongated device and operable to move the elongated device; and
a control unit configured to control the one or more servos according to the measurement provided by the signal analyzer.

5. An apparatus according to claim 4, wherein the controller is further configured to transmit a control signal to the power source according to the measurement provided by the signal analyzer.

6. An apparatus according to claim 3, further comprising a control unit configured to control one or more servos according to the measurement provided by the signal analyzer.

7. An apparatus according to claim 3, wherein the measurement indicative of an extent of cauterization comprises one of impedance and anti-resonance frequency.

8. An apparatus according to claim 1, further comprising:
a sensor operable to sense a property of tissue in contact with the sensor; and
a signal analyzer electrically coupled to the sensor and operable to determine one or more tissue boundaries from the tissue property sensed by the sensor.

9. An apparatus according to claim 1, further comprising:
a piezoelectric sensor integrated with the elongated device; and
a signal analyzer electrically coupled to the piezoelectric sensor, the signal analyzer configured to provide a measurement indicative of an extent of cauterization sensed by the piezoelectric sensor.

10. An apparatus according to claim 1, wherein the piezoelectric transducer comprises one or more lead zirconate titanate (PZT) discs.

11. An apparatus according to claim 10, wherein the one or more PZT discs are mounted in a cavity formed in a wall of the elongated device.

12. An apparatus according to claim 11, wherein the one or more PZT discs are mounted in a cavity formed in a wall of the elongated device.

13. An apparatus according to claim 12, wherein a wall of the cavity acts as a diaphragm of the piezoelectric transducer.

14. A method for obtaining a biopsy, comprising:
inserting a biopsy needle in a patient, the biopsy needle including a piezoelectric transducer mounted on the biopsy needle;
obtaining a biopsy from target tissue with the biopsy needle;
using a power source electrically coupled to the piezoelectric transducer to cause the piezoelectric transducer to generate heat to cauterize tissue; and
extracting the biopsy needle.

15. A method according to claim 14, wherein a sensor is mounted to the biopsy needle, wherein the sensor is adapted to sense properties of tissue proximate to the sensor, wherein the method further comprises:
monitoring tissue properties sensed by the sensor to determine an extent of cauterization.

16. A method according to claim 15, further comprising monitoring tissue properties sensed by the sensor to determine one or more tissue boundaries.

17. A method according to claim 15, wherein the monitoring step, further comprising:
mechanically coupling the biopsy needle to a servo;
coupling a control unit to one or both of the servo and the power source; and
implementing in the control unit a control algorithm operable to monitor the extent of the cauterization and to control either or both of the servo and the power source.

18. A method according to claim 15, wherein the monitored tissue property is one of impedance and anti-resonance frequency.

19. A method of cauterizing or ablating living tissue, comprising:
inserting an elongated medical device into a patient, the elongated device including a piezoelectric transducer mounted on the elongated device;
using a power source electrically coupled to the piezoelectric transducer to cause the piezoelectric transducer to generate heat to cauterize or ablate the tissue; and
extracting the elongated medical device.

20. A method according to claim 19, wherein the elongated medical device is a medical probe.

21. A method according to claim 19, wherein the elongated medical device is a needle.

22. A method according to claim 19, wherein a sensor is mounted to the elongated medical device, the method further comprising:
sensing a property of tissue proximate to the sensor; and
determining from the sensed property an extent of cauterization or ablation.

23. A method according to claim 19, further comprising:
mechanically coupling the elongated medical device to a servo;
coupling a control unit to one or both of the servo and the power source; and
implementing in the control unit a control algorithm operable to monitor the extent of the cauterization and to control either or both of the servo and the power source.
